(19) United States
(12) Patent Application Publication
(10) Pub. No.: US 2020/0006413 A1
(43) Pub. Date: Jan. 2, 2020

(54) PHOTOELECTRIC CONVERSION DEVICE, ELECTRONIC APPARATUS, AND METHOD FOR MANUFACTURING PHOTOELECTRIC CONVERSION DEVICE

(71) Applicant: SEIKO EPSON CORPORATION, Tokyo (JP)

(72) Inventor: Yasunori HATTORI, Shiojiri-shi (JP)

(73) Assignee: SEIKO EPSON CORPORATION, Tokyo (JP)

(21) Appl. No.: 16/458,777

(22) Filed: Jul. 1, 2019

(30) Foreign Application Priority Data
Jul. 2, 2018 (JP) 2018-125818

(51) Int. Cl. H01L 27/146; H01L 31/0224; H01L 31/032; H01L 29/24; H01L 29/786

(52) U.S. Cl. CPC H01L 27/14616; H01L 27/1461; H01L 27/14649; H01L 27/14689; A61B 5/14532; H01L 31/0322; H01L 29/24; H01L 29/7869; H01L 31/022475

(57) ABSTRACT

A photoelectric conversion device includes: a photoelectric conversion section containing an oxide semiconductor; and a transistor provided corresponding to the photoelectric conversion section, wherein a semiconductor layer of the transistor is made of the same material as that of the oxide semiconductor.

The present application is based on, and claims priority from JP Application Serial Number 2018-125818, filed Jul. 2, 2018, the disclosure of which is hereby incorporated by reference herein in its entirety.

### BACKGROUND

### 1. Technical Field

The present disclosure relates to a photoelectric conversion device, an electronic apparatus, and a method for manufacturing a photoelectric conversion device.

### 2. Related Art

An optical sensor in which photodiodes are disposed in a two-dimensional matrix has been widely used. This optical sensor is referred to as a photoelectric conversion device. JP-A-2012-169517 discloses a photoelectric conversion device. According to JP-A-2012-169517, the photoelectric conversion device includes a plurality of photodiodes that convert light into electric signals. A transistor is placed in each of the photodiodes. The transistor functions as a switching element that switches the photodiode to output a signal.

In the photodiode, a first electrode, a light absorbing layer, an oxide semiconductor layer, a window layer, and a second electrode are stacked in this order. The first electrode is a molybdenum film. The light absorbing layer is a CIGS (Cu(Inₓ, Ga₁₋ₓ)Se₂)-based film of a chalcopyrite structure. The oxide semiconductor layer is a film of IGZO (InGaZnO). In the window layer, a zinc oxide film and a zinc oxide film doped with an n-type impurity are stacked. The second electrode is a transparent electrode.

The transistor has a structure in which a gate insulating film and a gate electrode are disposed at an n-type semiconductor film. An electrode is formed in a source-drain region of the n-type semiconductor film. The gate insulating film is a silicon dioxide film. The gate electrode is a film of aluminum. JP-A-2010-205798 discloses that an amorphous oxide semiconductor of IGZO attracts attention as a semiconductor film of a transistor.

JP-A-2012-169517 and JP-A-2010-205798 are examples of the related art.

In the photoelectric conversion device disclosed in JP-A-2012-169517, a layer constituting the photodiode and a layer constituting the transistor are formed of different material. Hence, when the layers are formed, the layers are formed in different apparatuses in the manufacturing step. Therefore, a photoelectric conversion device of a structure that can be manufactured with better productivity is desired.

### SUMMARY

A photoelectric conversion device according to an aspect of the present application includes: a photoelectric conversion section containing an oxide semiconductor; and a transistor provided corresponding to the photoelectric conversion section, wherein a semiconductor layer of the transistor is made of the same material as that of the oxide semiconductor.

In the photoelectric conversion device, the photoelectric conversion section may include a first electrode, a p-type semiconductor layer, an n-type semiconductor layer containing the oxide semiconductor, and a second electrode, and the photoelectric conversion device may include the transistor including a gate electrode made of the same material as that of the first electrode and a source-drain electrode made of the same material as that of the second electrode.

In the photoelectric conversion device, the photoelectric conversion section may include an insulating film provided so as to cover a side surface of the p-type semiconductor layer, and the transistor may include a gate insulating film made of the same material as that of the insulating film.

In the photoelectric conversion device, the n-type semiconductor layer may contain an amorphous semiconductor.

In the photoelectric conversion device, the oxide semiconductor may be an oxide containing In, Ga, and Zn.

In the photoelectric conversion device, material of the first electrode may be Mo, and material of the second electrode may be ITO.

In the photoelectric conversion device, the p-type semiconductor layer may be Cu[Inₓ, Ga₁₋ₓ]Se₂, x being greater than or equal to 0 and less than or equal to 1.

An electronic apparatus according to an aspect of the present application includes the photoelectric conversion device according to the above aspect.

A method for manufacturing a photoelectric conversion device according to an aspect of the present application is a method for manufacturing a photoelectric conversion device including a photoelectric conversion section containing an oxide semiconductor and a transistor including a semiconductor layer containing the oxide semiconductor, the method including forming the oxide semiconductor of the photoelectric conversion section and the semiconductor layer in the same step.

In the method for manufacturing the photoelectric conversion device, the photoelectric conversion section may include a first electrode, a p-type semiconductor layer, an n-type semiconductor layer containing the oxide semiconductor, and a second electrode, the first electrode and a gate electrode of the transistor may be formed in the same step, and the second electrode and a source-drain electrode may be formed in the same step.

In the method for manufacturing the photoelectric conversion device, an insulating film covering a side surface of the p-type semiconductor layer and a gate insulating film of the transistor may be formed in the same step.

### BRIEF DESCRIPTION OF THE DRAWINGS

FIG. 1 is a schematic wiring diagram showing the configuration of a photoelectric conversion device according to a first embodiment.

FIG. 2 is an equivalent circuit diagram showing the configuration of a photosensor.

FIG. 3 is a main part schematic plan view showing the configuration of the photosensor.

FIG. 4 is a main part schematic sectional side view showing the configuration of the photosensor.

FIG. 5 is a flowchart of a method for manufacturing the photosensor.

FIG. 6 is a schematic view for explaining the method for manufacturing the photosensor.

FIG. 7 is a schematic view for explaining the method for manufacturing the photosensor.

FIG. 8 is a schematic view for explaining the method for manufacturing the photosensor.

FIG. 9 is a schematic view for explaining the method for manufacturing the photosensor.

FIG. 10 is a schematic view for explaining the method for manufacturing the photosensor.

FIG. 11 is a schematic view for explaining the method for manufacturing the photosensor.

FIG. 12 is a schematic view for explaining the method for manufacturing the photosensor.

FIG. 13 is a schematic view for explaining the method for manufacturing the photosensor.

FIG. 14 is a schematic view for explaining the method for manufacturing the photosensor.

FIG. 15 is a main part schematic sectional side view showing the configuration of a photosensor according to a second embodiment.

FIG. 16 is a flowchart of a method for manufacturing the photosensor.

FIG. 17 is a schematic view for explaining the method for manufacturing the photosensor.

FIG. 18 is a schematic view for explaining the method for manufacturing the photosensor.

FIG. 19 is a schematic perspective view showing the configuration of a biological information acquisition device according to a third embodiment.

FIG. 20 is a block diagram showing the electrical configuration of the biological information acquisition device.

### DESCRIPTION OF EXEMPLARY EMBODIMENTS

Hereinafter, embodiments will be described according to the drawings. Members in the drawings are illustrated on different scales so that each of the members has a recognizable size in the drawings.

### First Embodiment

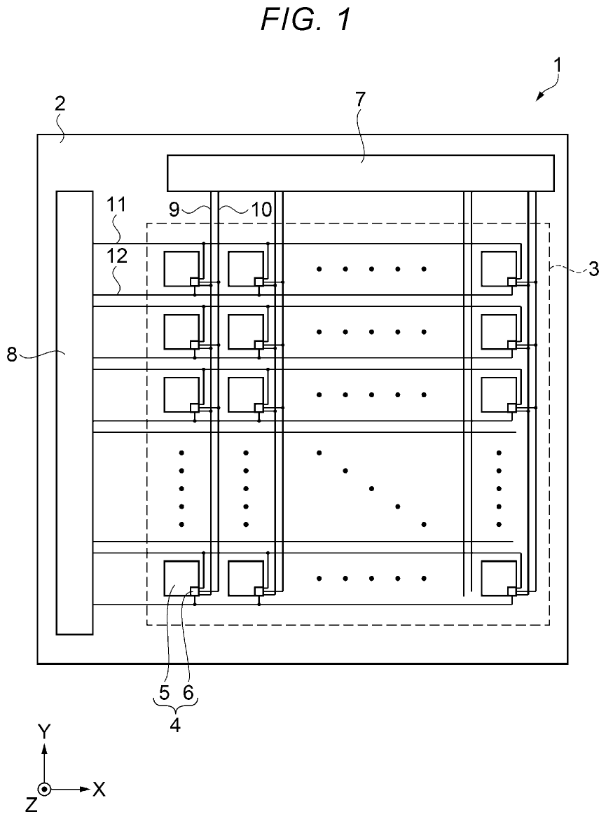

In a first embodiment, distinctive examples of a photoelectric conversion device and a method for manufacturing the photoelectric conversion device will be described according to the drawings. The photoelectric conversion device according to the first embodiment will be described according to FIGS. 1 to 4. FIG. 1 is a schematic wiring diagram showing the configuration of the photoelectric conversion device. The photoelectric conversion device 1 shown in FIG. 1 is a device on which light is incident and which converts a distribution of light into electric signals.

The photoelectric conversion device 1 includes a substrate 2. An element region 3 is set in the substrate 2. A plurality of photosensors 4 are disposed in a two-dimensional matrix in the element region 3. The directions in which the photosensors 4 are arrayed are defined as an X-direction and a Y-direction. The thickness direction of the substrate 2 is defined as a Z-direction. The X-direction, the Y-direction, and the Z-direction are directions orthogonal to each other.

The photosensor 4 includes a photoelectric conversion section 5 and a transistor 6. The photoelectric conversion section 5 is a photodiode on which light is incident and which causes an electric current corresponding to the intensity of incident light to flow. The transistor 6 functions as a switch to switch whether or not to output an output of the photodiode. As described above, the transistor 6 is provided corresponding to the photoelectric conversion section 5.

A data line drive circuit 7 is disposed at the +Y-direction side of the element region 3. A scanning line drive circuit 8 is disposed at the −X-direction side of the element region 3. The Y-direction in which the photosensors 4 are arrayed is defined as a column direction, and the X-direction in which the photosensors 4 are arrayed is defined as a row direction. A data wiring line 9 and a first potential wiring line 10 are disposed between the data line drive circuit 7 and each of the photosensors 4. The data wiring line 9 and the first potential wiring line 10 are disposed parallel to each other. A column of photosensors 4 arranged in the column direction is electrically coupled with the same data wiring line 9. The column of photosensors 4 arranged in the column direction is electrically coupled with the same first potential wiring line 10.

A second potential wiring line 11 and a scanning wiring line 12 are disposed between the scanning line drive circuit 8 and each of the photosensors 4. The second potential wiring line 11 and the scanning wiring line 12 are disposed parallel to each other. A row of photosensors 4 arranged in the row direction is electrically coupled through the same second potential wiring line 11. The row of photosensors 4 arranged in the row direction is electrically coupled through the same scanning wiring line 12. Voltages at the first potential wiring line 10 and the second potential wiring line 11 are constant voltages. The voltage at the first potential wiring line 10 is a voltage higher than the voltage at the second potential wiring line 11.

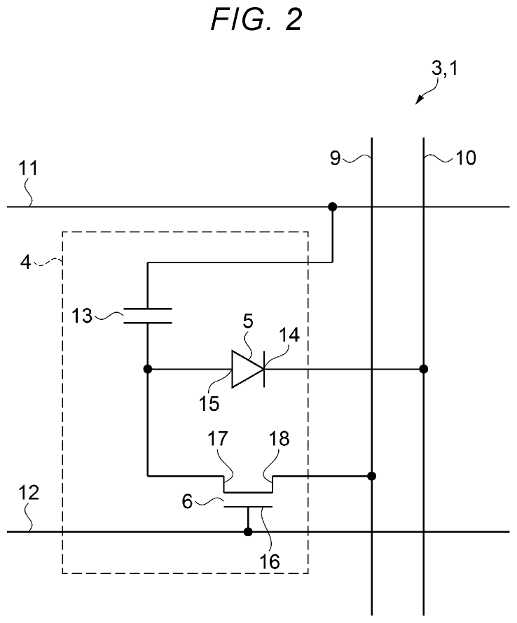

FIG. 2 is an equivalent circuit diagram showing the configuration of the photosensor. As shown in FIG. 2, the photosensor 4 includes the photoelectric conversion section 5, the transistor 6, and a storage capacitor 13. The transistor 6 is also referred to as a thin film transistor (TFT) element. A second electrode 14 of the photoelectric conversion section 5 is electrically coupled with the first potential wiring line 10. The second electrode 14 is also referred to as a cathode electrode. A first electrode 15 of the photoelectric conversion section 5 is electrically coupled with one electrode of the storage capacitor 13. The first electrode 15 is also referred to as an anode electrode. The other electrode of the storage capacitor 13 is electrically coupled with the second potential wiring line 11.

The first electrode 15 of the photoelectric conversion section 5 is electrically coupled with a first source-drain electrode 17 serving as a source-drain electrode belonging to the transistor 6. A second source-drain electrode 18 serving as a source-drain electrode belonging to the transistor 6 is electrically coupled with the data wiring line 9. Agate electrode 16 of the transistor 6 is electrically coupled with the scanning wiring line 12.

A voltage higher than that of the first electrode 15 is applied to the second electrode 14 of the photoelectric conversion section 5. Hence, a reverse bias voltage is applied to the photoelectric conversion section 5. When light is incident on the photoelectric conversion section 5, an electric current corresponding to the intensity of light flows through the photoelectric conversion section 5. The electric current corresponding to the intensity of light is referred to as a photocurrent. A charge according to the photocurrent is accumulated in the storage capacitor 13.

The scanning line drive circuit 8 applies a voltage signal of a pulse waveform to the gate electrode 16 of the transistor 6 via the scanning wiring line 12. The pulse waveform is normally maintained at a low voltage. The voltage of the pulse waveform is increased only in a predetermined period. At this time, an electric current flows between the first source-drain electrode 17 and the second source-drain electrode 18. Then, a signal of a voltage corresponding to the charge accumulated in the storage capacitor 13 is output to the data wiring line 9. The scanning line drive circuit 8 successively switches the voltage of the scanning wiring line 12 in each row. With this configuration, signals of voltages corresponding to the charges accumulated in the storage capacitors 13 of the photosensors 4 in each row are successively output to the data wiring line 9.

The data wiring line 9 in each column is electrically coupled to the data line drive circuit 7. Signals of voltages are simultaneously output to the data line drive circuit 7 from a plurality of the photosensors 4 in a row in which the voltage of the scanning wiring line 12 is increased by the scanning line drive circuit 8. In this manner, the photoelectric conversion device 1 can output a distribution of light detected by the photosensors 4.

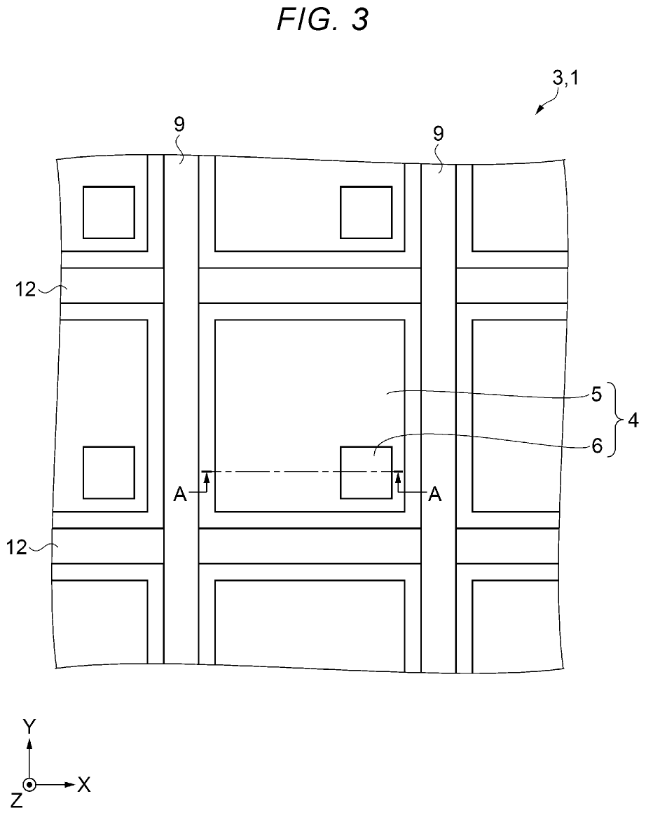

FIG. 3 is a main part schematic plan view showing the configuration of the photosensor. As shown in FIG. 3, the data wiring lines 9 are disposed at equal intervals in the X-direction. The scanning wiring lines 12 are disposed at equal intervals in the Y-direction. The data wiring lines 9 and the scanning wiring lines 12 are disposed in a grid-like manner. The photosensor 4 is disposed between the data wiring lines 9 and the scanning wiring lines 12. The photoelectric conversion section 5 and the transistor 6 are disposed in the photosensor 4. An area occupied by the transistor 6 is an area narrower than that of the photoelectric conversion section 5.

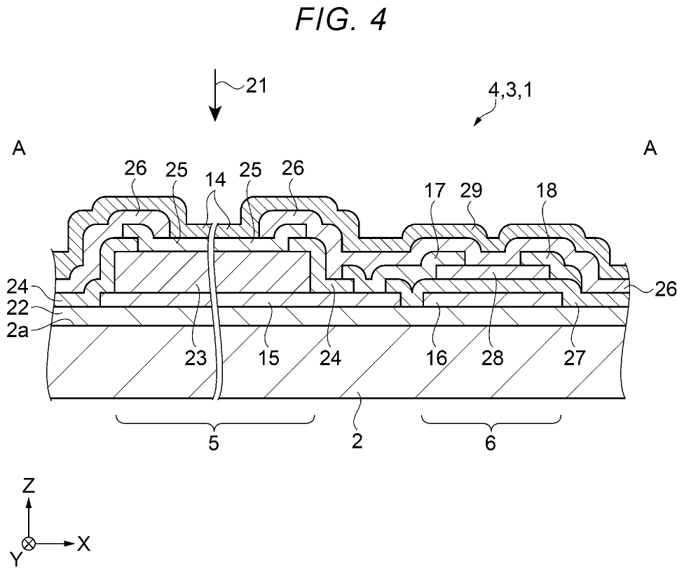

FIG. 4 is a main part schematic sectional side view showing the configuration of the photosensor as viewed from a surface side along line A-A in FIG. 3. As shown in FIG. 4, the photosensor 4 includes the photoelectric conversion section 5 and the transistor 6, which are provided at a side of a surface 2a of the substrate 2. Light 21 is incident on the photoelectric conversion section 5 from the +Z-direction side.

It is sufficient that the material of the substrate 2 has rigidity and heat resistance. For example, a glass substrate, a quartz substrate, or the like can be used as the substrate 2. In the embodiment, for example, a glass substrate is used as the substrate 2. A first insulating film 22 is formed at the surface 2a of the substrate 2. The first insulating film 22 prevents an electric signal of the photoelectric conversion section 5 or the transistor 6 from leaking into the substrate 2.

The first electrode 15 is formed in an island shape at the first insulating film 22 in the photoelectric conversion section 5. It is sufficient that the material of the first electrode 15 is metal having heat resistance. For example, metal material such as molybdenum (Mo), niobium (Nb), tantalum (Ta), or tungsten (W) can be used as the material of the first electrode 15. In the embodiment, for example, the material of the first electrode 15 is molybdenum (Mo). Molybdenum has a melting point as high as 2610° C. and has heat resistance. For this reason, the melting of the first electrode 15 can be prevented even when the photoelectric conversion section 5 is formed at a high temperature. The thickness of the first electrode 15 is not particularly limited, and in the embodiment, the thickness is set to, for example, substantially 400 nm.

A p-type semiconductor layer 23 is formed at the first electrode 15. The p-type semiconductor layer 23 functions as a light absorbing layer. A CIS-based (CuInSe₂, CuInGaSe, etc.) thin film of a chalcopyrite structure can be used as the p-type semiconductor layer 23. In the embodiment, for example, the p-type semiconductor layer 23 is Cu[Inₓ, Ga₁₋ₓ]Se₂, x being greater than or equal to 0 and less than or equal to 1. By changing the p-type semiconductor layer 23 from Cu(InGa)Se₂ to CuInSe₂, the wavelength range of light receivable by the photosensor 4 can be extended to substantially 1300 nm, which is the wavelength of near-infrared light. In this manner, the p-type semiconductor layer 23 can absorb near-infrared light. Hence, the photoelectric conversion device 1 can detect near-infrared light.

A second insulating film 24 serving as an insulating film is formed to cover the outer periphery of a surface of the +Z-direction side of the p-type semiconductor layer 23 and a side surface thereof. The second insulating film 24 is made of silicon dioxide (SiO₂). Hence, the photoelectric conversion section 5 includes the second insulating film 24 provided so as to cover the side surface of the p-type semiconductor layer 23. The second insulating film 24 covers a portion of the first electrode 15. Further, the second insulating film 24 also covers a portion of the first insulating film 22.

The second insulating film 24 is opened in the surface of the +Z-direction side of the p-type semiconductor layer 23. An n-type semiconductor layer 25 is formed in contact with the surface of the +Z-direction side of the p-type semiconductor layer 23. The n-type semiconductor layer 25 contains an amorphous oxide semiconductor. The amorphous oxide semiconductor preferably contains a Group 12 element or a Group 13 element defined by the International Union of Pure and Applied Chemistry (IUPAC). In the embodiment, a-IGZO (InGaZnO) is used for the n-type semiconductor layer 25. The letter “a” of the word “a-IGZO” represents the word “amorphous”. Amorphous means amorphous. The n-type semiconductor layer 25 is a so-called IGZO film containing indium (In), gallium (Ga), zinc (Zn), and oxygen (O). Hence, the oxide semiconductor contained in the n-type semiconductor layer 25 is an oxide containing In, Ga, and Zn. The photoelectric conversion section 5 contains the oxide semiconductor.

A third insulating film 26 is formed to cover the outer periphery of a surface of the +Z-direction side of the n-type semiconductor layer 25 and a side surface thereof. The third insulating film 26 is composed of a silicon nitride film (SiN). The silicon nitride film has a high effect of blocking impurity ions and therefore prevents variations in the characteristics of the photoelectric conversion section 5 due to impurity ions.

The third insulating film 26 is opened in the surface of the +Z-direction side of the n-type semiconductor layer 25. The second electrode 14 is formed in contact with the surface of the +Z-direction side of the n-type semiconductor layer 25. It is sufficient that the kind of material of the second electrode 14 is transparent and has conductivity, and the kind of material of the second electrode 14 is not particularly limited. For example, indium-gallium oxide (IGO), indium-tin oxide (ITO), indium-cerium oxide (ICO), or the like can be used as the material of the second electrode 14. In the embodiment, for example, the material of the second electrode 14 is ITO. Indium-tin oxide transmits the light 21, and therefore, the photoelectric conversion device 1 can efficiently take in the light 21.

As described above, the photoelectric conversion section 5 includes the first electrode 15, the p-type semiconductor layer 23, the n-type semiconductor layer 25 containing the oxide semiconductor, and the second electrode 14. The first electrode 15 is electrically coupled with the second potential wiring line 11 via the storage capacitor 13. The second electrode 14 is electrically coupled with the first potential wiring line 10. Hence, the potential of the second electrode 14 is a potential higher than the potential of the first electrode 15.

When the photoelectric conversion section 5 is irradiated with the light 21, electrons are excited in the p-type semiconductor layer 23 and the n-type semiconductor layer 25, and free electrons and free holes are generated. The free electrons generated in the p-type semiconductor layer 23 flow to the n-type semiconductor layer 25. The free holes generated in the p-type semiconductor layer 23 remain in the p-type semiconductor layer 23. The free electrons generated in the n-type semiconductor layer 25 remain in the n-type semiconductor layer 25. The free holes generated in the n-type semiconductor layer 25 flow to the p-type semiconductor layer 23. As a result, free holes increase in the p-type semiconductor layer 23, and free electrons increase in the n-type semiconductor layer 25. Then, an electric current flows from the first electrode 15 to the storage capacitor 13, and a charge is accumulated in the storage capacitor 13. The p-type semiconductor layer 23 is also referred to as a light absorbing layer.

The gate electrode 16 is formed in an island shape at the first insulating film 22 in the transistor 6. The gate electrode 16 is made of the same material as that of the first electrode 15. That is, the material of the gate electrode 16 and the first electrode 15 is molybdenum. Hence, the first electrode 15 of the photoelectric conversion section 5 and the gate electrode 16 of the transistor 6 can be configured without separately using other material. Moreover, the first electrode 15 of the photoelectric conversion section 5 and the gate electrode 16 of the transistor 6 can be manufactured in the same step using the same apparatus. That is, deposition and patterning can be performed. In this case, the numbers of deposition steps and patterning steps can be reduced compared to those when the first electrode 15 of the photoelectric conversion section 5 and the gate electrode 16 of the transistor 6 are manufactured respectively in separate steps.

Agate insulating film 27 is formed to cover the gate electrode 16. In the transistor 6, the gate insulating film 27 is made of the same material as that of the second insulating film 24. That is, the material of the gate insulating film 27 and the second insulating film 24 is silicon dioxide. Hence, the second insulating film 24 of the photoelectric conversion section 5 and the gate insulating film 27 of the transistor 6 can be manufactured in the same step using the same apparatus. That is, deposition and patterning can be performed. In this case, the numbers of deposition steps and patterning steps can be reduced compared to those when the second insulating film 24 of the photoelectric conversion section 5 and the gate insulating film 27 of the transistor 6 are manufactured respectively in separate steps. As a result, the photoelectric conversion device 1 can be manufactured with good productivity.

At a surface of the +Z-direction side of the gate insulating film 27, a semiconductor layer 28 is formed at a place opposed to the gate electrode 16. The semiconductor layer 28 of the transistor 6 contains an oxide semiconductor. The semiconductor layer 28 of the transistor 6 is made of a-IGZO, which is the same material as that of the oxide semiconductor of the n-type semiconductor layer 25. Hence, the transistor 6 includes the semiconductor layer 28 containing the oxide semiconductor. In the configuration of the photosensor 4, the oxide semiconductor of the photoelectric conversion section 5 and the semiconductor layer 28 of the transistor 6 can be configured without separately using other material. Moreover, the oxide semiconductor of the photoelectric conversion section 5 and the semiconductor layer 28 of the transistor 6 can be manufactured in the same step using the same apparatus. That is, deposition and patterning can be performed. In this case, the numbers of deposition steps and patterning steps can be reduced compared to those when the oxide semiconductor of the photoelectric conversion section 5 and the semiconductor layer 28 of the transistor 6 are manufactured respectively in separate steps. As a result, the photoelectric conversion device 1 can be manufactured with good productivity.

The n-type semiconductor layer 25 and the semiconductor layer 28 contain the amorphous semiconductor. The n-type semiconductor layer 25 of the amorphous semiconductor tends to have less leakage current compared to that when the n-type semiconductor layer 25 is not the amorphous semiconductor. Hence, the switching characteristics of the transistor 6 can be improved.

The n-type semiconductor layer 25 and the semiconductor layer 28 contain the oxide semiconductor. This oxide semiconductor is a-IGZO and is the oxide containing In, Ga, and Zn. In this case, the oxide semiconductor can act as the semiconductor layer 28 of the transistor 6.

At the −X-direction side of the semiconductor layer 28, the first source-drain electrode 17 is formed in contact with a surface of the +Z-direction side of the semiconductor layer 28. The first source-drain electrode 17 is disposed up to the first electrode 15 toward the −X-direction side at the gate insulating film 27. Hence, the first source-drain electrode 17 is electrically coupled with the first electrode 15.

At the +X-direction side of the semiconductor layer 28, the second source-drain electrode 18 is formed in contact with the surface of the +Z-direction side of the semiconductor layer 28. The second source-drain electrode 18 is disposed at the gate insulating film 27. The second source-drain electrode 18 is electrically coupled with the data wiring line 9.

The first source-drain electrode 17 and the second source-drain electrode 18 are made of the same material as that of the second electrode 14. That is, the material of the first source-drain electrode 17 and the second source-drain electrode 18 is ITO. Hence, the second electrode 14 of the photoelectric conversion section 5, and the first source-drain electrode 17 and the second source-drain electrode 18 can be configured without separately using other material. Moreover, the second electrode 14 of the photoelectric conversion section 5, and the first source-drain electrode 17 and the second source-drain electrode 18 can be manufactured using the same apparatus. That is, deposition and patterning can be performed.

The transistor 6 includes the gate electrode 16, the gate insulating film 27, and the semiconductor layer 28. The first source-drain electrode 17 and the second source-drain electrode 18 are not contained in the transistor 6. The first source-drain electrode 17 and the second source-drain electrode 18 are coupled to the transistor 6 and cause electricity to flow thereto.

The third insulating film 26 is formed at surfaces of the +Z-direction side of the first source-drain electrode 17, the second source-drain electrode 18, and the semiconductor layer 28. This third insulating film 26 is the same film as the third insulating film 26 formed in the photoelectric conversion section 5. The third insulating film 26 is composed of a silicon nitride film (SiN). The silicon nitride film has a high effect of blocking impurity ions and therefore prevents variations in the characteristics of the transistor 6 due to impurity ions.

A wiring line 29 is formed at a surface of the +Z-direction side of the third insulating film 26. The wiring line 29 electrically couples the second electrode 14 with the first potential wiring line 10. Further, an insulating film may be disposed to cover the second electrode 14 and the wiring line 29.

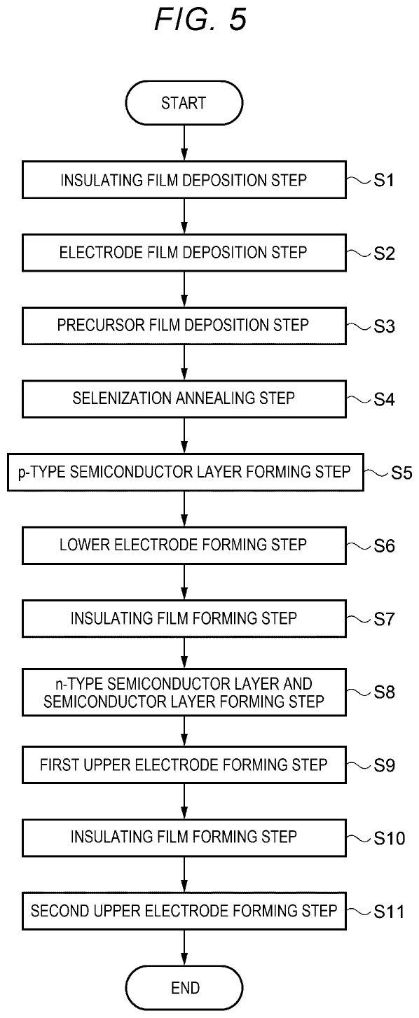

Next, a method for manufacturing the photoelectric conversion device 1 described above will be described. In the method for manufacturing the photoelectric conversion device 1, a method for forming the data line drive circuit 7, the scanning line drive circuit 8, the data wiring line 9, the first potential wiring line 10, the second potential wiring line 11, and the scanning wiring line 12 at the substrate 2 is publicly known, and the description of the forming method is omitted. A method for manufacturing the photosensor 4 will be described with reference to FIGS. 5 to 14.

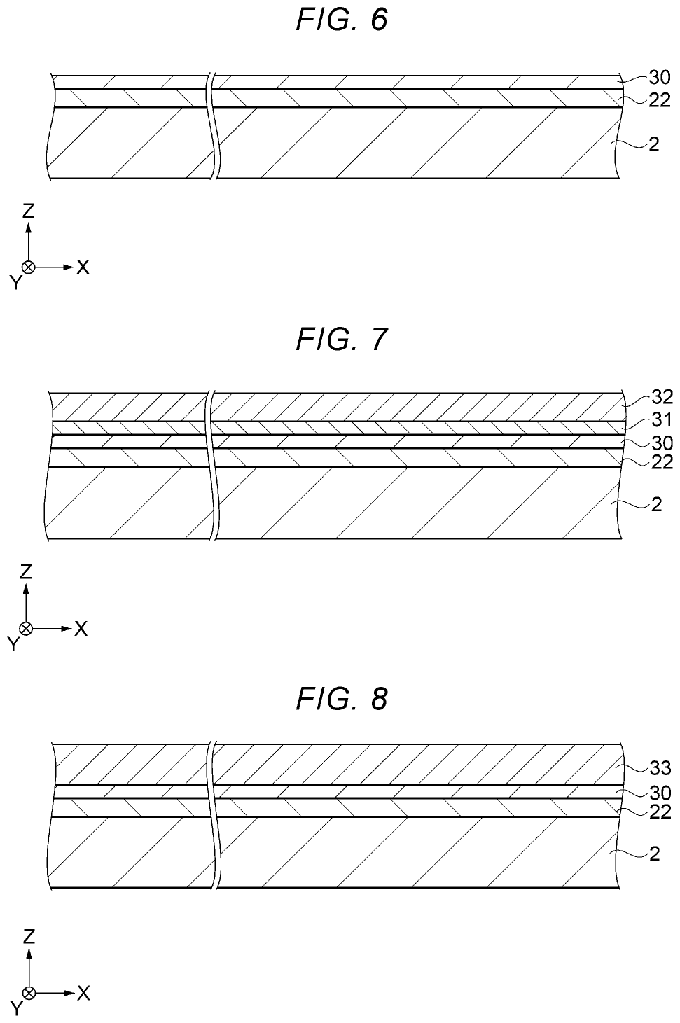

FIG. 5 is a flowchart of the method for manufacturing the photosensor, and FIGS. 6 to 14 are schematic views for explaining the method for manufacturing the photosensor. In the flowchart of FIG. 5, Step S1 corresponds to an insulating film deposition step and is a step for depositing the first insulating film 22 at the substrate 2. Next, the method proceeds to Step S2. Step S2 is an electrode film deposition step. This step is a step for depositing a metal film serving as the base of the first electrode 15 and the gate electrode 16 at the first insulating film 22. Next, the method proceeds to Step S3. Step S3 is a precursor film deposition step. This step is a step for depositing a film serving as the precursor of the p-type semiconductor layer 23 at the metal film. Next, the method proceeds to Step S4.

Step S4 is a selenization annealing step. This step is a step for subjecting the film serving as the precursor of the p-type semiconductor layer 23 to heat treatment in a hydrogen selenide atmosphere. Next, the method proceeds to Step S5. Step S5 is a p-type semiconductor layer forming step. This step is a step for patterning the p-type semiconductor layer 23 into a predetermined shape. Next, the method proceeds to Step S6. Step S6 is a lower electrode forming step. This step is a step for patterning the first electrode 15 and the gate electrode 16 into predetermined shapes. Next, the method proceeds to Step S7.

Step S7 is an insulating film forming step. This step is a step for depositing the second insulating film 24 and the gate insulating film 27 and patterning the second insulating film 24 and the gate insulating film 27 into predetermined shapes. Next, the method proceeds to Step S8. Step S8 is an n-type semiconductor layer and semiconductor layer forming step. This step is a step for depositing the n-type semiconductor layer 25 and the semiconductor layer 28 and patterning the n-type semiconductor layer 25 and the semiconductor layer 28 into predetermined shapes. Next, the method proceeds to Step S9. Step S9 is a first upper electrode forming step. This step is a step for depositing an ITO film and patterning the ITO film into the shapes of the first source-drain electrode 17 and the second source-drain electrode 18. Next, the method proceeds to Step S10.

Step S10 is an insulating film forming step. This step is a step for depositing the third insulating film 26 and patterning the third insulating film 26 into a predetermined shape. Next, the method proceeds to Step S11. Step S11 is a second upper electrode forming step. This step is a step for depositing an ITO film and patterning the ITO film into the shapes of the second electrode 14, the wiring line 29, and the like. Through the steps described above, a step for manufacturing the photosensor 4 is finished.

Next, with reference to FIGS. 6 to 14, the method for manufacturing the photosensor 4 will be described in detail in correspondence with Steps shown in FIG. 5.

FIG. 6 is a diagram corresponding to the insulating film deposition step of Step S1 and the electrode film deposition step of Step S2. As shown in FIG. 6, the substrate 2 is prepared in Step S1. The substrate 2 is alkali-free glass not containing alkali metal. Hence, the substrate 2 can prevent alkali metal from being contained in the p-type semiconductor layer 23.

The first insulating film 22 is deposited at the substrate 2. The first insulating film 22 is a film of silicon dioxide. The first insulating film 22 is deposited using a plasma-enhanced chemical vapor deposition (CVD) method.

In Step S2, a molybdenum film 30 is deposited at the first insulating film 22. The molybdenum film 30 is a film whose material is molybdenum. The molybdenum film 30 is deposited using a sputtering method. The thickness of the molybdenum film 30 is substantially 400 nm.

FIG. 7 is a diagram corresponding to the precursor film deposition step of Step S3. As shown in FIG. 7, in Step S3, the film serving as the precursor of the p-type semiconductor layer 23 is deposited at the molybdenum film 30. The film serving as a precursor is also referred to as a precursor film. First, an alloy film 31 containing copper (Cu) and gallium (Ga) is deposited at the molybdenum film 30. Further, an indium film 32 containing indium (In) is deposited at the alloy film 31. The alloy film 31 and the indium film 32 are deposited using a sputtering method. The sum of the thickness of the alloy film 31 and the thickness of the indium film 32 is substantially 1.5 μm.

FIG. 8 is a diagram corresponding to the selenization annealing step of Step S4. As shown in FIG. 8, a p-type semiconductor film 33 that is a film serving as the base of the p-type semiconductor layer 23 is formed from the alloy film 31 and the indium film 32. Heat treatment is applied to the alloy film 31 and the indium film 32 in an atmosphere containing a Group 16 element. In the embodiment, hydrogen selenide (H₂Se) is used as gas containing a Group 16 element, and heat treatment is applied at, for example, a temperature of substantially from 400° C. to 500° C. The concentration of hydrogen selenide is adjusted within from 1 to 20%. This heat treatment is treatment for allowing the alloy film 31 and the indium film 32 to react with a Group 16 element to form the p-type semiconductor film 33 of a chalcopyrite structure.

By applying heat treatment to the alloy film 31 and the indium film 32, the p-type semiconductor film 33 of a chalcopyrite structure is formed. In the embodiment, by applying heat treatment in a hydrogen selenide atmosphere, the alloy film 31 (Cu, Ga) and the indium film 32 (In) are selenized, and the p-type semiconductor film 33 composed of a CIGS (Cu(In, Ga)Se₂)-based film is formed.

Moreover, hydrogen sulfide (H₂S) may be used as gas containing a Group 16 element for an atmosphere when heat treatment is applied, and heat treatment maybe further applied in an H₂S atmosphere after heat treatment is applied in a hydrogen selenide atmosphere.

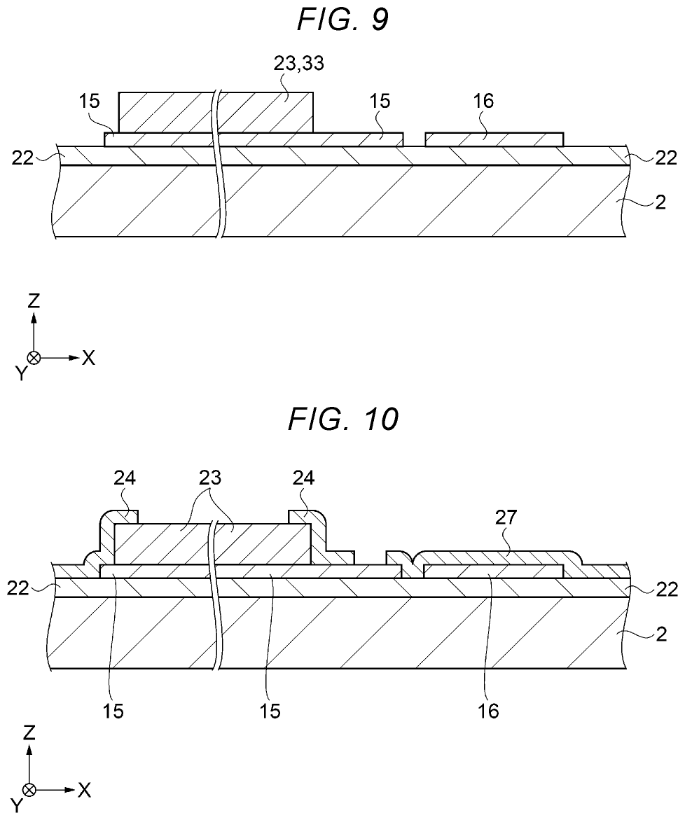

FIG. 9 is a diagram corresponding to the p-type semiconductor layer forming step of Step S5 and the lower electrode forming step of Step S6. As shown in FIG. 9, in Step S5, the p-type semiconductor film 33 is patterned into the shape of the p-type semiconductor layer 23. A lithography method and a dry etching method are used as the patterning method.

Specifically, a mask film is placed at the p-type semiconductor film 33. First, the material of the mask film is applied to the p-type semiconductor film 33. The material of the mask film is obtained by dissolving photosensitive resin material in a solvent. Various coating methods or printing methods can be used as the application method. Next, the material of the mask film is dried. Subsequently, exposure and development are performed, and a film made of the material of the mask film is patterned to form the mask film. Then, the shape of the mask film is made into the shape of the p-type semiconductor layer 23. Next, the p-type semiconductor film 33 is processed into the shape of the mask film using ions or radicals generated by producing plasma within a chamber of an apparatus.

In Step S6, the molybdenum film 30 is patterned into the shapes of the first electrode 15 and the gate electrode 16. A lithography method and a dry etching method are used as the patterning method. Hence, the first electrode 15 and the gate electrode 16 of the transistor 6 are formed in the same step. That is, in Step S2, the deposition of the molybdenum film 30 serving as the base of the first electrode 15 and the gate electrode 16 is performed in the same step. Then, in Step S6, the patterning of the first electrode 15 and the gate electrode 16 is performed in the same step. In this case, the numbers of deposition steps and patterning steps can be reduced compared to those when the first electrode 15 of the photoelectric conversion section 5 and the gate electrode of the transistor 6 are manufactured respectively in separate steps.

FIG. 10 is a diagram corresponding to the insulating film forming step of Step S7. As shown in FIG. 10, in Step S7, the second insulating film 24 and the gate insulating film 27 are formed. The second insulating film 24 and the gate insulating film 27 are films of silicon dioxide. In this step, a film of silicon dioxide is first deposited. A plasma CVD method is used as the deposition method of the film of silicon dioxide. Next, the film of silicon dioxide is patterned into the shapes of the second insulating film 24 and the gate insulating film 27. A lithography method and a dry etching method are used as the patterning method.

The second insulating film 24 is an insulating film that covers the side surface of the p-type semiconductor layer 23. Hence, the second insulating film 24 covering the side surface of the p-type semiconductor layer 23 and the gate insulating film 27 of the transistor 6 are formed in the same step. That is, the deposition and patterning of the second insulating film 24 and the gate insulating film 27 are performed in the same step. In this case, the numbers of deposition steps and patterning steps can be reduced compared to those when the second insulating film 24 of the photoelectric conversion section 5 and the gate insulating film 27 of the transistor 6 are manufactured respectively in separate steps. As a result, the photoelectric conversion device 1 can be manufactured with good productivity.

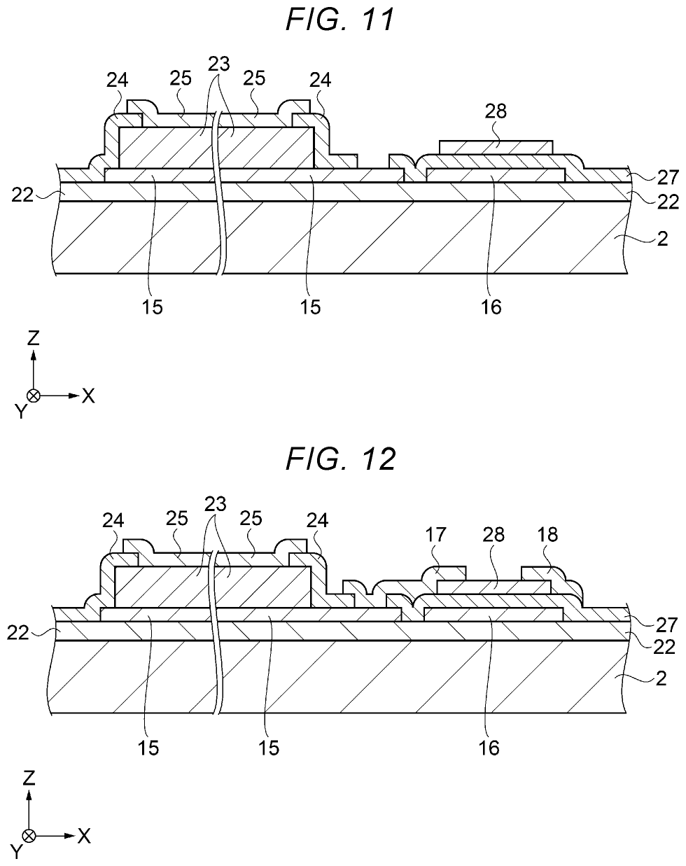

FIG. 11 is a diagram corresponding to the n-type semiconductor layer and semiconductor layer forming step of Step S8. As shown in FIG. 11, in Step S8, the n-type semiconductor layer 25 and the semiconductor layer 28 are formed. The material of the n-type semiconductor layer 25 and the semiconductor layer 28 is InGaZnO. First, a film of InGaZnO is deposited. A sputtering method is used as the deposition method of the film of InGaZnO. In addition, a pulsed laser deposition (PLD) method may be used. The film is deposited by depositing InGaZnO at the p-type semiconductor layer 23 and the gate insulating film 27 using as a target a sintered body of a three-component oxide containing InGaZnO₄ or In₂O₃—Ga₂O₃—ZnO.

When deposition is performed, an oxygen partial pressure of an atmosphere within a deposition chamber is set to a proper range. The oxygen partial pressure represents a partial pressure of oxygen gas intentionally introduced into the deposition chamber. A channel layer can be formed in the semiconductor layer 28 by controlling the oxygen partial pressure of the atmosphere within the deposition chamber to set a residual electron carrier concentration to from 10¹⁵ to 10²⁰ cm⁻³. Next, water vapor is mixed in the chamber in which the film of InGaZnO is placed, and the film of InGaZnO is heated in an oxygen atmosphere for substantially one hour. A heat treatment temperature is preferably substantially from 350 to 450° C., and a dew point is preferably substantially from 40 to 80° C.

Next, the film of InGaZnO is patterned into the shapes of the n-type semiconductor layer 25 and the semiconductor layer 28. A lithography method and a dry etching method are used as the patterning method.

As described above, the n-type semiconductor layer 25 of the photoelectric conversion section 5, which contains the oxide semiconductor, and the semiconductor layer 28 are formed in the same step. That is, deposition and patterning can be performed. In this case, the numbers of deposition steps and patterning steps can be reduced compared to those when the n-type semiconductor layer 25 of the photoelectric conversion section 5 and the semiconductor layer 28 of the transistor 6 are manufactured respectively in separate steps. As a result, the photoelectric conversion device 1 can be manufactured with good productivity.

FIG. 12 is a diagram corresponding to the first upper electrode forming step of Step S9. As shown in FIG. 12, the first source-drain electrode 17 and the second source-drain electrode 18 are formed. The material of the first source-drain electrode 17 and the second source-drain electrode 18 is ITO. First, an ITO film is deposited. A sputtering method is used as the deposition method of the ITO film. Next, the ITO film is patterned into the shapes of the first source-drain electrode 17 and the second source-drain electrode 18. A lithography method and a dry etching method are used as the patterning method.

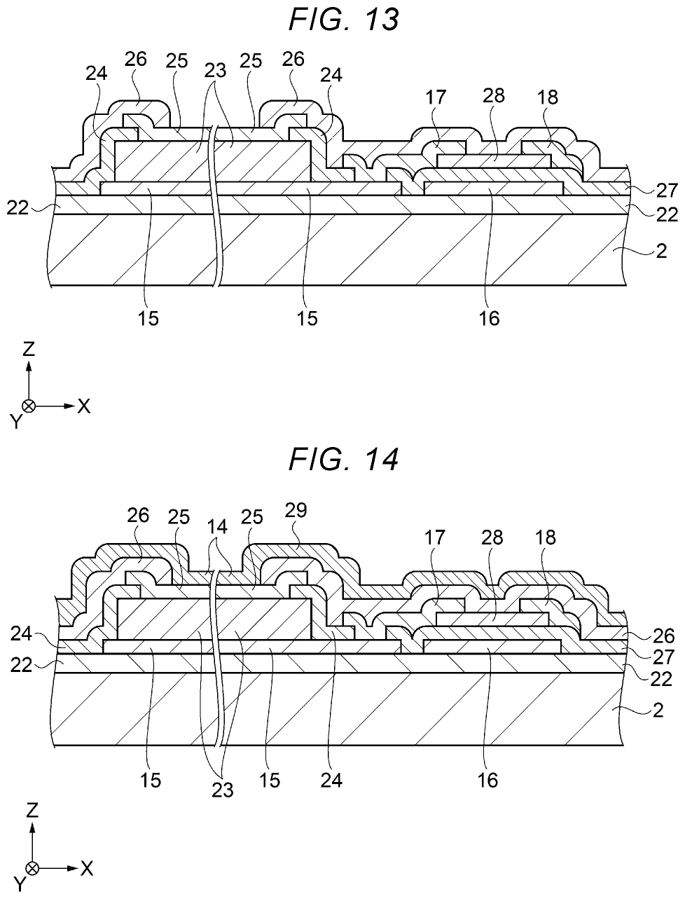

FIG. 13 is a diagram corresponding to the insulating film forming step of Step S10. As shown in FIG. 13, the third insulating film 26 is formed to cover the peripheries of the semiconductor layer 28, the first source-drain electrode 17, the second source-drain electrode 18, the second insulating film 24, and the n-type semiconductor layer 25. The third insulating film 26 is a silicon nitride film. First, the silicon nitride film is deposited. A plasma CVD method is used as the deposition method of the silicon nitride film. Next, the silicon nitride film is patterned into the shape of the third insulating film 26. In this case, the third insulating film 26 at a portion facing the n-type semiconductor layer 25 is partially opened to expose the n-type semiconductor layer 25. A lithography method and a dry etching method are used as the patterning method.

FIG. 14 is a diagram corresponding to the second upper electrode forming step of Step S11. As shown in FIG. 14, the second electrode 14 is formed to be electrically coupled with the n-type semiconductor layer 25 exposed from the third insulating film 26. Further, the wiring line 29 is formed to be electrically coupled with the second electrode 14 at the third insulating film 26. Further, the wiring line 29 is electrically coupled with the first potential wiring line 10. The material of the second electrode 14 and the wiring line 29 is ITO. First, an ITO film is deposited. A sputtering method is used as the deposition method of the ITO film. Next, the ITO film is patterned into the shapes of the second electrode 14 and the wiring line 29. A lithography method and a dry etching method are used as the patterning method. Through the steps described above, the step for manufacturing the photosensor 4 is finished.

As described above, according to the embodiment, the following advantageous effects are provided.

(1) According to the embodiment, the photoelectric conversion device 1 includes the photoelectric conversion section 5 and the transistor 6. The photoelectric conversion section 5 contains the oxide semiconductor. The transistor includes the semiconductor layer 28. The oxide semiconductor and the semiconductor layer 28 are made of the same IGZO.

Hence, the oxide semiconductor of the photoelectric conversion section 5 and the semiconductor layer 28 of the transistor 6 can be configured without separately using other material. Moreover, the oxide semiconductor of the photoelectric conversion section 5 and the semiconductor layer 28 of the transistor 6 can be manufactured in the same step using the same apparatus. That is, deposition and patterning can be performed. In this case, the numbers of deposition steps and patterning steps can be reduced compared to those when the oxide semiconductor of the photoelectric conversion section 5 and the semiconductor layer 28 of the transistor 6 are manufactured respectively in separate steps. As a result, the photoelectric conversion device 1 can be manufactured with good productivity.

(2) According to the embodiment, the photoelectric conversion section 5 includes the first electrode 15, the p-type semiconductor layer 23, the n-type semiconductor layer 25 containing the oxide semiconductor, and the second electrode 14. The first electrode 15 and the gate electrode 16 of the transistor 6 are made of the same molybdenum. The second electrode 14, the first source-drain electrode 17, and the second source-drain electrode 18 are made of the same ITO.

Hence, the first electrode 15 of the photoelectric conversion section 5 and the gate electrode 16 of the transistor 6 can be configured without separately using other material. Moreover, the first electrode 15 of the photoelectric conversion section 5 and the gate electrode 16 of the transistor 6 can be manufactured in the same step using the same apparatus. That is, deposition and patterning can be performed. In this case, the numbers of deposition steps and patterning steps can be reduced compared to those when the first electrode 15 of the photoelectric conversion section 5 and the gate electrode of the transistor 6 are manufactured respectively in separate steps.

(3) According to the embodiment, the second insulating film 24 is provided so as to cover the side surface of the p-type semiconductor layer 23 in the photoelectric conversion section 5. The second insulating film 24 and the gate insulating film 27 of the transistor 6 are made of the same silicon dioxide. Hence, the second insulating film 24 of the photoelectric conversion section 5 and the gate insulating film 27 of the transistor 6 can be manufactured in the same step using the same apparatus. That is, deposition and patterning can be performed. In this case, the numbers of deposition steps and patterning steps can be reduced compared to those when the second insulating film 24 of the photoelectric conversion section 5 and the gate insulating film of the transistor 6 are manufactured respectively in separate steps. As a result, the photoelectric conversion device 1 can be manufactured with good productivity.

(4) According to the embodiment, the n-type semiconductor layer 25 and the semiconductor layer 28 contain the amorphous semiconductor. The n-type semiconductor layer 25 of the amorphous semiconductor tends to have less leakage current compared to that when the n-type semiconductor layer 25 is not the amorphous semiconductor. Hence, the switching characteristics of the transistor 6 can be improved.

(5) According to the embodiment, the oxide semiconductor of the n-type semiconductor layer 25 is the oxide containing In, Ga, and Zn. In this case, the oxide semiconductor can act as the semiconductor layer 28 of the transistor 6.

(6) According to the embodiment, the material of the first electrode 15 is molybdenum, and the material of the second electrode 14 is ITO. Molybdenum has heat resistance, and therefore, the first electrode 15 has heat resistance. Hence, in the step for forming the p-type semiconductor layer 23 at the first electrode 15, the precursor of the p-type semiconductor layer 23 can be annealed at a high temperature. Indium-tin oxide, which is the material of the second electrode 14, is light transmissive. Hence, the photoelectric conversion device 1 can efficiently take in the light 21.

(7) According to the embodiment, the p-type semiconductor layer 23 is Cu[Inₓ, Ga₁₋ₓ]Seₑ, x being greater than or equal to 0 and less than or equal to 1. In this case, the p-type semiconductor layer 23 can absorb near-infrared light. Hence, the photoelectric conversion device 1 can detect near-infrared light.

(8) According to the embodiment, the photoelectric conversion device 1 includes the photoelectric conversion section 5 containing the oxide semiconductor and the transistor 6 containing the oxide semiconductor. The oxide semiconductor of the photoelectric conversion section 5 and the semiconductor layer 28 of the transistor 6 are formed in the same step. That is, deposition and patterning can be performed. In this case, the numbers of deposition steps and patterning steps can be reduced compared to those when the oxide semiconductor of the photoelectric conversion section 5 and the semiconductor layer of the transistor 6 are manufactured respectively in separate steps. As a result, the photoelectric conversion device 1 can be manufactured with good productivity.

(9) According to the embodiment, the photoelectric conversion section 5 includes the first electrode 15, the p-type semiconductor layer 23, the n-type semiconductor layer 25 containing the oxide semiconductor, and the second electrode 14. The first electrode 15 of the photoelectric conversion section 5 and the gate electrode 16 of the transistor 6 are disposed in the same step. That is, deposition and patterning are performed in the same step. In this case, the numbers of deposition steps and patterning steps can be reduced compared to those when the first electrode 15 of the photoelectric conversion section 5 and the gate electrode 16 of the transistor 6 are manufactured respectively in separate steps.

(10) According to the embodiment, the second insulating film 24 is provided so as to cover the side surface of the p-type semiconductor layer 23. The second insulating film 24 and the gate insulating film 27 of the transistor 6 are disposed in the same step. That is, deposition and patterning are performed in the same step. In this case, the numbers of deposition steps and patterning steps can be reduced compared to those when the second insulating film 24 of the photoelectric conversion section 5 and the gate insulating film of the transistor 6 are manufactured respectively in separate steps. As a result, the photoelectric conversion device 1 can be manufactured with good productivity.

### Second Embodiment

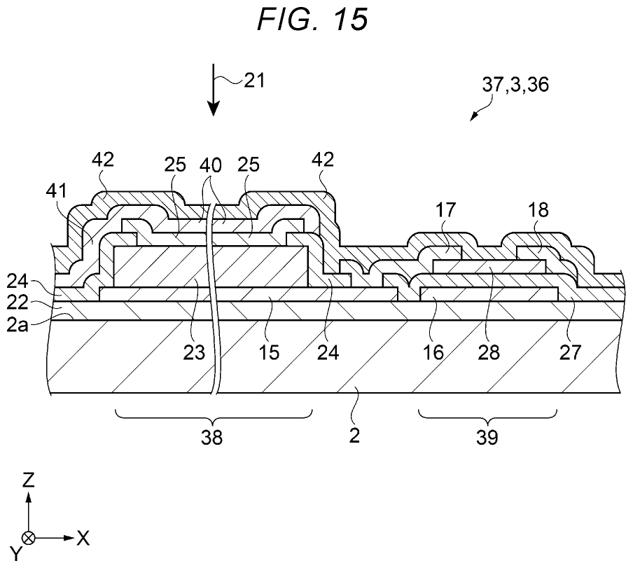

Next, one embodiment of a photoelectric conversion device will be described with reference to FIGS. 15 to 18. The embodiment differs from the first embodiment in that the arrangement of the second electrode 14 shown in FIG. 4 is different. The description of the same points as those of the first embodiment is omitted.

FIG. 15 is a main part schematic sectional side view showing the configuration of a photosensor. That is, in the embodiment, a photoelectric conversion device 36 includes the photosensor 37 in the element region 3 as shown in FIG. 15. A photoelectric conversion section 38 and a transistor 39 are disposed in the photosensor 37.

The arrangement of the first electrode 15, the p-type semiconductor layer 23, the second insulating film 24, and the n-type semiconductor layer 25 in the photoelectric conversion section 38 is the same arrangement as that in the photoelectric conversion section 5 in the first embodiment, and the description of the arrangement is omitted. A second electrode 40 is formed in contact with the surface of the +Z-direction side of the n-type semiconductor layer 25. The material of the second electrode 40 is ITO, which is the same as that of the second electrode 14 in the first embodiment. The first source-drain electrode 17 and the second source-drain electrode 18 are made of the same material as that of the second electrode 40.

A wiring line 41 is formed at a surface of the +Z-direction side of the second insulating film 24. The wiring line 41 electrically couples the second electrode 40 with the first potential wiring line 10.

The arrangement of the gate electrode 16, the gate insulating film 27, the semiconductor layer 28, the first source-drain electrode 17, and the second source-drain electrode 18 in the transistor 39 is the same arrangement as that in the transistor 6 in the first embodiment, and the description of the arrangement is omitted. A protective film 42 is formed to cover the photoelectric conversion section 38 and the transistor 39. The material of the protective film 42 is silicon nitride, which is the same as that of the third insulating film 26 in the first embodiment.

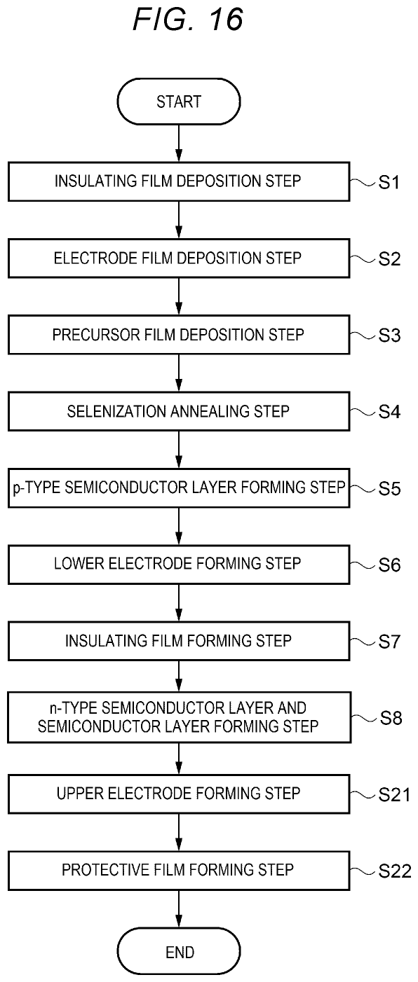

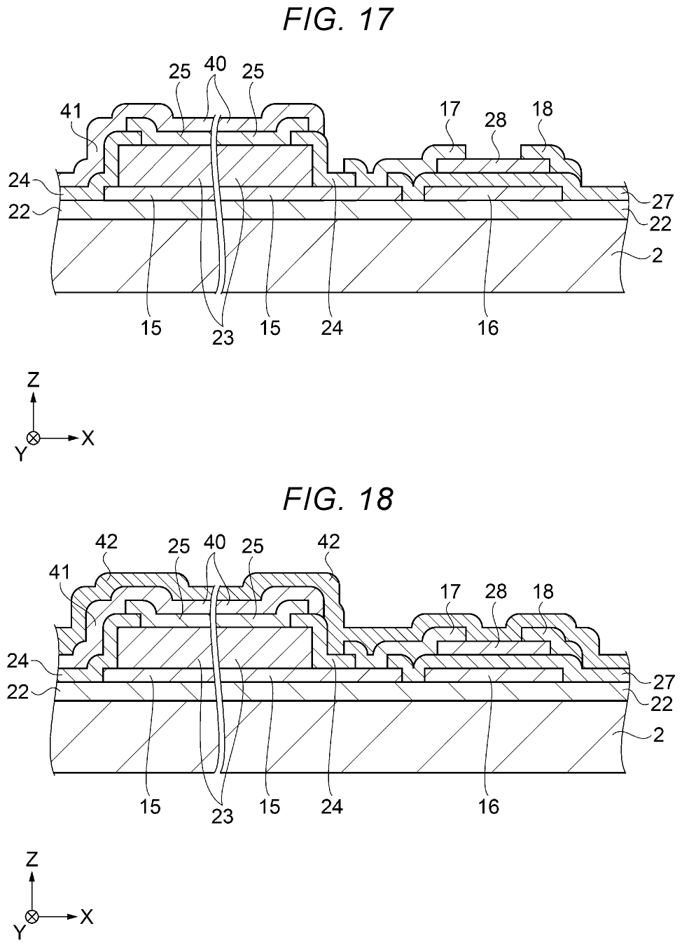

Next, a method for manufacturing the photosensor 37 of the photoelectric conversion device 36 described above will be described with reference to FIGS. 16 to 18. FIG. 16 is a flowchart of the method for manufacturing the photosensor, and FIGS. 17 and 18 are schematic views for explaining the method for manufacturing the photosensor. In the flowchart of FIG. 16, the insulating film deposition step of Step S1 to the n-type semiconductor layer and semiconductor layer forming step of Step S8 are the same steps as those in the first embodiment, and the description of the steps is omitted.

After Step S8, the method proceeds to Step S21. Step S21 is an upper electrode forming step. This step is a step for depositing an ITO film and patterning the ITO film into the shapes of the second electrode 40, the first source-drain electrode 17, and the second source-drain electrode 18. Next, the method proceeds to Step S22. Step S22 is a protective film forming step. This step is a step for depositing a silicon nitride film and patterning the silicon nitride film into a predetermined shape. Through the steps described above, a step for manufacturing the photosensor 37 is finished.

Next, with reference to FIGS. 17 and 18, the method for manufacturing the photosensor 37 will be described in detail in correspondence with Steps shown in FIG. 16.

FIG. 17 is a diagram corresponding to the upper electrode forming step of Step S21. As shown in FIG. 17, the second electrode 40, the wiring line 41, the first source-drain electrode 17, and the second source-drain electrode 18 are formed in Step S21.

The material of the second electrode 40, the wiring line 41, the first source-drain electrode 17, and the second source-drain electrode 18 is ITO. First, an ITO film is deposited. A sputtering method is used as the deposition method of the ITO film. Next, the ITO film is patterned into the shapes of the second electrode 40, the first source-drain electrode 17, and the second source-drain electrode 18. A lithography method and a dry etching method are used as the patterning method.

Hence, the second electrode 40, the first source-drain electrode 17, and the second source-drain electrode 18 are formed in the same step. That is, the deposition and patterning of the second electrode 40, the first source-drain electrode 17, and the second source-drain electrode 18 are performed in the same step. In this case, the numbers of deposition steps and patterning steps can be reduced compared to those when the second electrode 40 of the photoelectric conversion section 38, the first source-drain electrode 17, and the second source-drain electrode 18 are manufactured respectively in separate steps. As a result, the photoelectric conversion device 36 can be manufactured with good productivity.

FIG. 18 is a diagram corresponding to the protective film forming step of Step S22. As shown in FIG. 18, the protective film 42 is formed to cover the semiconductor layer 28, the first source-drain electrode 17, the second source-drain electrode 18, the second insulating film 24, and the second electrode 40. The protective film 42 is a silicon nitride film. First, the silicon nitride film is deposited. A plasma CVD method is used as the deposition method of the silicon nitride film. Next, the silicon nitride film is patterned into the shape of the protective film 42. A lithography method and a dry etching method are used as the patterning method. Through the steps described above, the step for manufacturing the photosensor 37 is finished.

As described above, according to the embodiment, the following advantageous effect is provided.

(1) According to the embodiment, the second electrode 40, the first source-drain electrode 17, and the second source-drain electrode 18 are formed in the same step. That is, deposition and patterning are performed in the same step. In this case, the numbers of deposition steps and patterning steps can be reduced compared to those when the second electrode 40 of the photoelectric conversion section 38, the first source-drain electrode 17, and the second source-drain electrode 18 are manufactured respectively in separate steps. As a result, the photoelectric conversion device 36 can be manufactured with good productivity.

### Third Embodiment

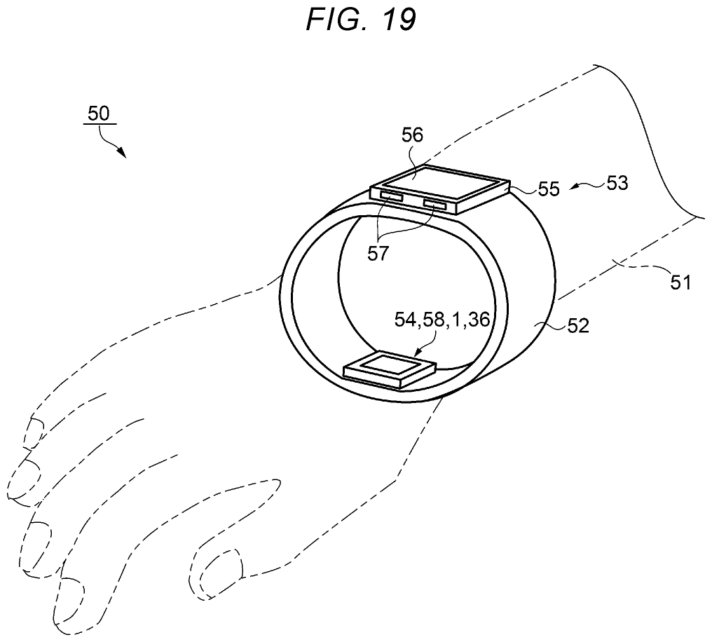

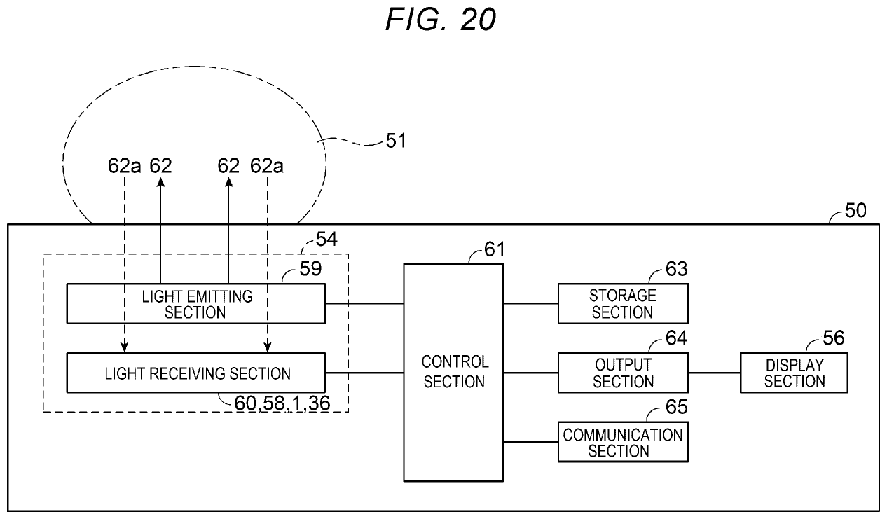

Next, one embodiment of an electronic apparatus in which the photoelectric conversion device 1 or the photoelectric conversion device 36 is mounted will be described with reference to FIGS. 19 and 20. FIG. 19 is a schematic perspective view showing the configuration of a biological information acquisition device. FIG. 20 is a block diagram showing the electrical configuration of the biological information acquisition device.

As shown in FIG. 19, the biological information acquisition device 50 as an electronic apparatus is a portable information terminal device worn on a wrist 51 of a human body. The biological information acquisition device 50 determines the position of a blood vessel in a living body from image information of the blood vessel inside the wrist 51. In addition, the biological information acquisition device 50 noninvasively detects optically the amount of a specific component, for example glucose or the like, contained in blood of a blood vessel to determine a blood-sugar level.

The biological information acquisition device 50 includes a belt 52, a main body section 53, and a sensor section 54. The belt 52 is annular and wearable on the wrist 51. The main body section 53 is attached to the outer surface of the belt 52. The sensor section 54 is attached to the inner surface of the belt 52 and disposed at a position opposed to the main body section 53.

The main body section 53 includes a main body case 55. A display section 56 is incorporated into the main body case 55. In addition, operating buttons 57, circuit system components of a control section and the like, a battery, and the like are incorporated into the main body case 55.

The sensor section 54 includes an image sensor 58. The sensor section 54 is electrically coupled with the main body section 53 through a wiring line incorporated into the belt 52. The image sensor 58 of the biological information acquisition device 50 includes the photoelectric conversion device 1 or the photoelectric conversion device 36. The photoelectric conversion device 1 and/or the photoelectric conversion device 36 can be manufactured with good productivity. Hence, the biological information acquisition device 50 can be an apparatus including the photoelectric conversion device 1 or the photoelectric conversion device 36, which can be manufactured with good productivity.

The biological information acquisition device 50 is worn for use such that the sensor section 54 is in contact with the wrist 51 at the hand palm side. By wearing the biological information acquisition device 50 in this manner, variations in detection sensitivity due to incidence of external light on the sensor section 54 or the skin of the wrist 51 can be avoided.

In the biological information acquisition device 50, the main body section 53 and the sensor section 54 are configured to be separately incorporated into the belt 52. However, the main body section 53 and the sensor section 54 may be configured to be integrated together, and the integrated one may be incorporated into the belt 52.

As shown in FIG. 20, the biological information acquisition device 50 includes a control section 61, the sensor section 54 electrically coupled to the control section 61, a storage section 63, an output section 64, and a communication section 65. Moreover, the biological information acquisition device 50 includes the display section 56 electrically coupled with the output section 64.

The sensor section 54 includes a light emitting section 59 and a light receiving section 60. The light emitting section 59 and the light receiving section 60 are each electrically coupled with the control section 61. The light emitting section 59 includes a light source section that emits near-infrared light 62. The wavelength of the near-infrared light 62 is within the range of from 700 nm to 2000 nm. The control section 61 drives the light emitting section 59 to cause the light emitting section 59 to emit the near-infrared light 62. The near-infrared light 62 propagates and scatters inside the wrist 51. The light receiving section 60 receives portion of the near-infrared light 62 scattering inside the wrist 51 as reflected light 62a.

The light receiving section 60 of the biological information acquisition device 50 includes the image sensor 58, and the photoelectric conversion device 1 or the photoelectric conversion device 36 is used for the image sensor 58.

The biological information acquisition device 50 includes the storage section 63, the output section 64, and the communication section 65. The control section 61 causes the storage section 63 to store information of the reflected light 62a received by the light receiving section 60. Then, the control section 61 causes the output section 64 to process the information of the reflected light 62a. The output section 64 converts the information of the reflected light 62a into image information of a blood vessel and outputs the image information. In addition, the output section 64 converts the information of the reflected light 62a into content information of a specific component in blood. In addition, the control section 61 causes the display section 56 to display the converted image information of the blood vessel or the converted information of the specific component in blood. The communication section 65 transmits these items of information to another information processor.

Moreover, the communication section 65 receives information of a program or the like from another information processor. Then, the control section 61 causes the storage section 63 to store the information of a program or the like. The display section 56 displays obtained information relating to a blood vessel or blood. In addition, the display section 56 displays the information of a program or the like previously stored in the storage section 63, or information of current time or the like.

As described above, according to the embodiment, the following advantageous effect is provided.

(1) According to the embodiment, the image sensor 58 of the biological information acquisition device 50 includes the photoelectric conversion device 1 or the photoelectric conversion device 36. The photoelectric conversion device 1 or the photoelectric conversion device 36 can be manufactured with good productivity. Hence, the biological information acquisition device 50 can be an apparatus including the photoelectric conversion device 1 or the photoelectric conversion device 36, which can be manufactured with good productivity.

The embodiment is not limited to the embodiment described above, and various modifications or improvements can be added within the technical idea of the present disclosure by a person having ordinary knowledge in the art. A modified example will be described below.

### MODIFIED EXAMPLE 1

In the third embodiment, the biological information acquisition device 50 including the photoelectric conversion device 1 or the photoelectric conversion device 36 has been described. In addition, the photoelectric conversion device 1 or the photoelectric conversion device 36 may be used for an imaging device that takes a picture of a fingerprint, an iris, a vein pattern, or the like. The photoelectric conversion device 1 or the photoelectric conversion device 36 can be manufactured with good productivity, and therefore, the imaging device can be a device including the photoelectric conversion device 1 or the photoelectric conversion device 36, which can be manufactured with good productivity.

Contents derived from the embodiments will be described below.

A photoelectric conversion device includes: a photoelectric conversion section containing an oxide semiconductor; and a transistor provided corresponding to the photoelectric conversion section, wherein a semiconductor layer of the transistor is made of the same material as that of the oxide semiconductor.

According to this configuration, the photoelectric conversion device includes the photoelectric conversion section and the transistor. The photoelectric conversion section contains the oxide semiconductor. The transistor includes the semiconductor layer. The oxide semiconductor and the semiconductor layer are made of the same material.

Hence, the oxide semiconductor of the photoelectric conversion section and the semiconductor layer of the transistor can be configured without separately using other material. Moreover, the oxide semiconductor of the photoelectric conversion section and the semiconductor layer of the transistor can be manufactured in the same step using the same apparatus. That is, deposition and patterning can be performed. In this case, the numbers of deposition steps and patterning steps can be reduced compared to those when the oxide semiconductor of the photoelectric conversion section and the semiconductor layer of the transistor are manufactured respectively in separate steps. As a result, the photoelectric conversion device can be manufactured with good productivity.

In the photoelectric conversion device, the photoelectric conversion section may include a first electrode, a p-type semiconductor layer, an n-type semiconductor layer containing the oxide semiconductor, and a second electrode, and the photoelectric conversion device may include the transistor including a gate electrode made of the same material as that of the first electrode and a source-drain electrode made of the same material as that of the second electrode.

According to this configuration, the photoelectric conversion section includes the first electrode, the p-type semiconductor layer, the n-type semiconductor layer containing the oxide semiconductor, and the second electrode. The first electrode and the gate electrode of the transistor are made of the same material. The second electrode and the source-drain electrode are made of the same material.

Hence, the first electrode of the photoelectric conversion section and the gate electrode of the transistor can be configured without separately using other material. Moreover, the first electrode of the photoelectric conversion section and the gate electrode of the transistor can be manufactured in the same step using the same apparatus. That is, deposition and patterning can be performed. In this case, the numbers of deposition steps and patterning steps can be reduced compared to those when the first electrode of the photoelectric conversion section and the gate electrode of the transistor are manufactured respectively in separate steps.

Similarly, the second electrode of the photoelectric conversion section and the source-drain electrode can be configured without separately using other material. Moreover, the second electrode of the photoelectric conversion section and the source-drain electrode can be manufactured in the same step using the same apparatus. That is, deposition and patterning can be performed. In this case, the numbers of deposition steps and patterning steps can be reduced compared to those when the second electrode of the photoelectric conversion section and the source-drain electrode are manufactured respectively in separate steps. As a result, the photoelectric conversion device can be manufactured with good productivity.

In the photoelectric conversion device, the photoelectric conversion section may include an insulating film provided so as to cover a side surface of the p-type semiconductor layer, and the transistor may include a gate insulating film made of the same material as that of the insulating film.

According to this configuration, the insulating film is provided so as to cover the side surface of the p-type semiconductor layer in the photoelectric conversion section. The insulating film and the gate insulating film of the transistor are made of the same material. Hence, the insulating film of the photoelectric conversion section and the gate insulating film of the transistor can be manufactured in the same step using the same apparatus. That is, deposition and patterning can be performed. In this case, the numbers of deposition steps and patterning steps can be reduced compared to those when the insulating film of the photoelectric conversion section and the gate insulating film of the transistor are manufactured respectively in separate steps. As a result, the photoelectric conversion device can be manufactured with good productivity.

In the photoelectric conversion device, the n-type semiconductor layer may contain an amorphous semiconductor.

According to this configuration, the n-type semiconductor layer contains the amorphous semiconductor. The n-type semiconductor layer of the amorphous semiconductor tends to have less leakage current compared to that when the n-type semiconductor layer is not the amorphous semiconductor. Hence, the switching characteristics of the transistor can be improved.

In the photoelectric conversion device, the oxide semiconductor may be an oxide containing In, Ga, and Zn.

According to this configuration, the oxide semiconductor is the oxide containing In, Ga, and Zn. In this case, the oxide semiconductor can act as the semiconductor layer of the transistor.

In the photoelectric conversion device, material of the first electrode may be Mo, and material of the second electrode may be ITO.

According to this configuration, the material of the first electrode is Mo, and the material of the second electrode is ITO. Molybdenum has heat resistance, and therefore, the first electrode has heat resistance. Hence, in a step for forming the p-type semiconductor layer at the first electrode, a precursor of the p-type semiconductor layer can be annealed at a high temperature. Indium-tin oxide, which is the material of the second electrode, is light transmissive. Hence, the photoelectric conversion device can efficiently take in light.

In the photoelectric conversion device, the p-type semiconductor layer may be Cu[Inₓ, Ga₁₋ₓ]Se₂, x being greater than or equal to 0 and less than or equal to 1.

According to this configuration, the p-type semiconductor layer is Cu[Inₓ, Ga₁₋ₓ]Se₂, x being greater than or equal to 0 and less than or equal to 1. In this case, the p-type semiconductor layer can absorb near-infrared light. Hence, the photoelectric conversion device can detect near-infrared light.

An electronic apparatus includes the photoelectric conversion device described above.

According to this configuration, the electronic apparatus includes the photoelectric conversion device described above. The photoelectric conversion device described above can be manufactured with good productivity. Hence, the electronic apparatus can be an apparatus including the photoelectric conversion device, which can be manufactured with good productivity.

A method for manufacturing a photoelectric conversion device is a method for manufacturing a photoelectric conversion device including a photoelectric conversion section containing an oxide semiconductor and a transistor including a semiconductor layer containing the oxide semiconductor, the method including forming the oxide semiconductor of the photoelectric conversion section and the semiconductor layer in the same step.

According to this method, the photoelectric conversion device includes the photoelectric conversion section containing the oxide semiconductor and the transistor containing the oxide semiconductor. The oxide semiconductor of the photoelectric conversion section and the semiconductor layer of the transistor are disposed in the same step. That is, deposition and patterning can be performed. In this case, the numbers of deposition steps and patterning steps can be reduced compared to those when the oxide semiconductor of the photoelectric conversion section and the semiconductor layer of the transistor are manufactured respectively in separate steps. As a result, the photoelectric conversion device can be manufactured with good productivity.

In the method for manufacturing the photoelectric conversion device, the photoelectric conversion section may include a first electrode, a p-type semiconductor layer, an n-type semiconductor layer containing the oxide semiconductor, and a second electrode, the first electrode and a gate electrode of the transistor may be formed in the same step, and the second electrode and a source-drain electrode may be formed in the same step.

According to this method, the photoelectric conversion section includes the first electrode, the p-type semiconductor layer, the n-type semiconductor layer containing the oxide semiconductor, and the second electrode. The first electrode of the photoelectric conversion section and the gate electrode of the transistor are disposed in the same step. That is, deposition and patterning are performed in the same step. In this case, the numbers of deposition steps and patterning steps can be reduced compared to those when the first electrode of the photoelectric conversion section and the gate electrode of the transistor are manufactured respectively in separate steps.

Similarly, the second electrode of the photoelectric conversion section and the source-drain electrode are disposed in the same step. That is, deposition and patterning are performed in the same step. In this case, the numbers of deposition steps and patterning steps can be reduced compared to those when the second electrode of the photoelectric conversion section and the source-drain electrode are manufactured respectively in separate steps. As a result, the photoelectric conversion device can be manufactured with good productivity.

In the method for manufacturing the photoelectric conversion device, an insulating film covering a side surface of the p-type semiconductor layer and a gate insulating film of the transistor may be formed in the same step.

According to this method, the insulating film is provided so as to cover the side surface of the p-type semiconductor layer. The insulating film and the gate insulating film of the transistor are disposed in the same step. That is, deposition and patterning of the insulating film and the gate insulating film of the transistor are performed in the same step. In this case, the numbers of deposition steps and patterning steps can be reduced compared to those when the insulating film of the photoelectric conversion section and the gate insulating film of the transistor are manufactured respectively in separate steps. As a result, the photoelectric conversion device can be manufactured with good productivity.

What is claimed is:

1. A photoelectric conversion device comprising:
a photoelectric conversion section containing an oxide semiconductor; and
a transistor provided corresponding to the photoelectric conversion section, wherein
a semiconductor layer of the transistor is made of the same material as that of the oxide semiconductor.

2. The photoelectric conversion device according to claim 1, wherein
the photoelectric conversion section includes
a first electrode,
a p-type semiconductor layer,
an n-type semiconductor layer containing the oxide semiconductor, and
a second electrode, and
the photoelectric conversion device includes
the transistor including a gate electrode made of the same material as that of the first electrode and
a source-drain electrode made of the same material as that of the second electrode.

3. The photoelectric conversion device according to claim 2, wherein
the photoelectric conversion section includes an insulating film provided so as to cover a side surface of the p-type semiconductor layer, and
the transistor includes a gate insulating film made of the same material as that of the insulating film.

4. The photoelectric conversion device according to claim 2, wherein
the n-type semiconductor layer contains an amorphous semiconductor.

5. The photoelectric conversion device according to claim 3, wherein
the n-type semiconductor layer contains an amorphous semiconductor.

6. The photoelectric conversion device according to claim 1, wherein
the oxide semiconductor is an oxide containing In, Ga, and Zn.

7. The photoelectric conversion device according to claim 2, wherein
the oxide semiconductor is an oxide containing In, Ga, and Zn.

8. The photoelectric conversion device according to claim 3, wherein
the oxide semiconductor is an oxide containing In, Ga, and Zn.

9. The photoelectric conversion device according to claim 4, wherein
the oxide semiconductor is an oxide containing In, Ga, and Zn.

10. The photoelectric conversion device according to claim 2, wherein
material of the first electrode is Mo, and
material of the second electrode is ITO.

11. The photoelectric conversion device according to claim 2, wherein
the p-type semiconductor layer is Cu[Inₓ, Ga₁₋ₓ]Se₂, x being greater than or equal to 0 and less than or equal to 1.

12. An electronic apparatus comprising the photoelectric conversion device according to claim 1.

13. An electronic apparatus comprising the photoelectric conversion device according to claim 2.

14. An electronic apparatus comprising the photoelectric conversion device according to claim 3.

15. An electronic apparatus comprising the photoelectric conversion device according to claim 7.
